(19) United States
(12) Patent Application Publication
(10) Pub. No.: US 2026/0215938 A1
(43) Pub. Date: Jul. 30, 2026

(54) CUSTOMIZABLE SUSPENSION LINER, SYSTEM, AND METHOD FOR MAKING THE SAME

(71) Applicant: OSSUR ICELAND EHF, Reykjavik (IS)

(72) Inventors: Rowan CAIN, Reykjavik (IS); Andri ORRASON, Reykjavik (IS); Stefan Orn STEFANSSON, Reykjavik (IS); Sigurdur ASGEIRSSON, Reykjavik (IS); Christophe Guy LECOMTE, Reykjavik (IS)

(21) Appl. No.: 19/144,614

(22) PCT Filed: Jan. 4, 2024
(86) PCT No.: PCT/US2024/010256

§ 371 Date: Jun. 30, 2025

Related U.S. Application Data
(60) Provisional application No. 63/478,622, filed on Jan. 5, 2023.

(51) Int. Cl. A61F 2/80; A61F 2/50; A61F 2/60; A61F 2/78

(52) U.S. Cl. CPC A61F 2/80; A61F 2002/5052; A61F 2002/5056; A61F 2002/607; A61F 2002/7837

(57) ABSTRACT

A customized prosthetic suspension liner adapted to envelop a unique shape of an individual residual limb by casting the liner directly on a residual limb. The suspension liner has an inner layer with an inner periphery arranged to follow the contour of the residual limb, an outer layer that forms an outer periphery of the suspension liner having a shape different from that of the inner periphery, and a middle layer formed between the inner and outer layers that defines a variable thickness between proximal and distal end regions of the liner to accommodate the different shapes of the inner and outer peripheries and provide a customized interface between the residual limb and prosthetic socket.

### CROSS-REFERENCE TO RELATED APPLICATION

This application incorporates by reference U.S. provisional application No. 63/478,622, filed on Jan. 5, 2023, and claims the benefit of priority.

### FIELD OF THE DISCLOSURE

This disclosure relates to a customized prosthetic suspension liner adapted to envelop the unique shape of an individual residual limb, a system including the suspension liner, and a method for making the same.

### BACKGROUND

Prosthetic suspension liners have been described in prior patents, such as U.S. Pat. No. 4,923,474, issued May 8, 1990; U.S. Pat. No. 6,136,039, issued Oct. 24, 2000; U.S. Pat. No. 6,485,776, issued Nov. 26, 2002; U.S. Pat. No. 7,118,602, issued Oct. 10, 2006; and U.S. Pat. No. 7,169,189, issued on Jan. 30, 2007, each of which is incorporated herein by reference. These liners or sleeves may be fabricated of elastic or elasticized materials and are used to cushion a post-operative stump or residual limb concerning a prosthesis, such as a socket, that is installed over the residual limb and coupled to the liner or sleeve by a conventional locking element.

Suspension liners are commercially available and comprise a variety of sizes adapted to fit a wide range of residual limbs. A practitioner can select a size and type of suspension liner that most closely approximates the amputee's residual leg size. This generic silicone sleeve is designed primarily to allow for improved suspension (retention) of a prosthetic leg on a residual leg. These suspension liners should conform closely to the residual limb, accommodate all surface contours and sub-surface bone elements of the residual limb, and provide a comfortable cushion between the residual limb and the hard socket of the prosthesis fitted over the residual limb.

Certain residual limbs have a unique limb shape, and custom-shaped liners may be recommended for limbs having prominent bony limbs, limbs with sharp or irregular contours, a high level of sensitivity, or significant scarring, such as limbs with skin grafts. Custom liners require a prosthetist to fit the residual limb's unique limb shape to accommodate different lifestyles and activity levels while preserving maximum comfort and suspension.

Prior art methods for forming a customized suspension liner involve producing a mold of the amputee's residual leg to create a positive mold. Such a process is cumbersome, time-intensive, and costly, yet further limited to the efficacy of manufacturing the positive mold. The mold is sent to a laboratory that fabricates the customized liner based on the positive mold. It does not benefit from adaptation directly to the residual limb during manufacture.

While effective solutions have been proposed and implemented for making custom suspension liners, it is still highly desirable to improve the comfort of such liners or sleeves to increase their ability to conform to irregularities on a unique residual limb and provide an amputee with enhanced comfort at a residual limb interface with a prosthesis while maintaining sufficient strength and durability. Moreover, it is particularly desirable to provide a method for making a suspension liner that can be manufactured directly onto an amputee without the need for making a positive mold and thereby relying on the efficacy of the positive mold in that the suspension liner can be modified in situ on the residual limb itself. Further, customizing a suspension liner versus an off-the-shelf suspension liner may lead to more adoption of wearing the suspension liner since fit and comfort can be better confirmed.

For the preceding reasons, there is a need to provide improved liners that impart improved conformance, pressure distribution, comfort, and stretching while maintaining sufficient durability and strength for their requisite applications. There is a need to offer a method of manufacture that enables a prosthetist to fabricate such liner directly on the amputee to ensure comfort and fit as the liner is fabricated in a timely and cost-effective manner.

### SUMMARY

The system and method of the present invention overcome the problems above and allow the manufacture of a custom prosthetic liner in a timely and cost-efficient manner. Furthermore, the system and method of the present invention eliminate the need to cast an amputee's residual limb to obtain the proper shape thereof. Rather, embodiments of the liner, a prosthetic system including such liner, and methods of the liner can obtain the exact shape of an amputee's residual limb by casting the liner directly on a residual limb with a high degree of accuracy.

According to the customized method and subsequent liner, a practitioner can modify the residual limb model to fine-tune the fit of the liner further while worn on the user to obtain instant feedback on fit against a socket and the residual limb. Thus, the liner is customized for the individual residual limb. Such liner can be used in a prosthetic system, including at least the suspension liner and a socket, to offer improved fit and comfort. In addition, fabricating the liner eliminates a liner mold to produce the custom liner. It, therefore, eliminates the possibility of variability from the residual limb and a liner mold since the liner is directly cast on the user.

The method cuts the need to send a liner mold to a laboratory and lengthy lead times to completion, enabling the user to quickly use the liner once a moldable material forming a middle layer of the liner has cured. The practitioner can make sizing adjustments while the customized liner is worn to ensure fit and comfort. The method can be provided by a kit, enabling prefabrication of a liner blank before forming the definitive liner, enabling a commercial means for easily fabricating a customized prosthetic liner with minimal education or skill. The kit may include the liner blank, a sufficient quantity of moldable material, and tools and devices to inject the moldable material into the liner blank.

These and other features of the present disclosure will become better understood regarding the following description, appended claims, and accompanying drawings.

### BRIEF DESCRIPTION OF THE DRAWINGS

The numerous other advantages, features, and functions of embodiments of a suspension liner, prosthetic system, and method for making the same will become readily apparent and better understood given the following description and accompanying drawings. The following description is not intended to limit the scope of the suspension liner, prosthetic system, and method for making the same, but instead merely provides exemplary embodiments for ease of understanding.

FIG. 1A is a schematic view showing a residual limb wearing a liner blank before fabrication into a suspension liner and between a prosthetic socket.

FIG. 1B is a schematic view illustrating a gap between the liner blank and the socket of FIG. 1A.

FIG. 2A is a schematic view showing the residual limb of FIG. 1A after the suspension liner has been fabricated, the liner blank becoming the suspension liner upon fabrication.

FIG. 2B is a schematic view illustrating a fit between the suspension liner and the socket of FIG. 2A.

FIG. 3 illustrates a graphical representation of a custom liner on an off-the-shelf mold.

FIG. 4 illustrates a graphical representation of a custom liner on an off-the-shelf mold produced on a standard-shaped mandrel.

FIG. 5 is a schematic view illustrating fabricating the suspension liner by inserting moldable material into the liner blank.

FIG. 6 is a schematic view illustrating inserting the moldable material into the liner blank.

FIG. 7 is a schematic view illustrating an embodiment of a casting core.

FIG. 8 is a detailed view taken from FIG. 7 showing an exemplary pattern.

FIG. 9 is a variation of a distal end of the casting core.

FIG. 10 is an exemplary cross-sectional view of a filled suspension liner, according to FIG. 5.

FIG. 11 is a schematic view of a liner blank, including an orientation liner.

FIG. 12 is a schematic view showing a method of drawings air from a proximal end of a suspension liner during fabrication.

FIG. 13 is a schematic view of the suspension liner in FIG. 12.

FIG. 14 is a schematic view of a variation of fabricating a suspension liner in FIG. 6 without a socket disposed over the liner blank.

FIG. 15 is a schematic view of another method step according to FIG. 14.

FIG. 16 is a schematic view of a method of fabricating a suspension liner, including using a pressure band.

FIG. 17 is a schematic view of using pressure control pads in forming a socket for the method of FIG. 6.

FIG. 18 is another schematic view of FIG. 17.

FIG. 19 is a schematic view illustrating another embodiment of a casting core, including an injection tube.

FIG. 20 is a schematic view illustrating a liner blank formed by the casting core of FIG. 19 and injection of silicone from a proximal edge.

FIGS. 21 and 22 are schematic views showing an embodiment of first and second sides, respectively, of a soft umbrella.

FIGS. 23 and 24 are schematic views showing another embodiment of first and second sides, respectively, of a soft umbrella.

### DEFINITIONS

A description of a few terms is necessary for further ease of understanding the disclosure. As used herein, the term “proximal” has its ordinary meaning and refers to a location that is closer to the heart than another location. Likewise, the term “distal” has its ordinary meaning and refers to a location that is further from the heart than another location. The term “posterior” also has its ordinary meaning and refers to a location behind or another location's rear. Lastly, the term “anterior” has its ordinary meaning and refers to a location that is ahead of or to the front of another location.

The terms “rigid,” “flexible,” and “resilient” may be used herein to distinguish characteristics of portions of certain features of the prosthetic system. The term “rigid” denotes that an element of the system generally lacks flexibility. On the other hand, the term “flexible” denotes that features can repeat bending. The features may be bent into retained shapes, or the features do not retain a general shape but continuously deform when force is applied. Finally, “resilient” qualifies such flexible features as returning to an initial general shape without permanent deformation. The term “semi-rigid” is used to connote properties of elements that provide support and are free-standing; however, such elements may have some degree of flexibility or resiliency.

The term “bond” or “bonding” refers to any type of heat bonding, induction bonding, or chemical bonding between two materials, such as between the moldable material and the casting core, according to the chemical and physical features of the material of the customized suspension liner. By chemical bonding, a preferred embodiment is without an adhesive, in that the liner materials are bonded together as the moldable material is a curing material and sufficiently fluid for the layers to at least slightly blend at an interface between inner and outer layers; however it is not outside the scope of the disclosure to use an adhesive, a primer, or any other suitable attachment means.

The term “compatible” refers to a material that does not react with, break down, or absorb another material in an unintended way and does not impair the chemical and/or mechanical properties of the other material in an unintended way. For example, the term may refer to a blend of materials with sufficient interphase mixing and favorable interaction between the materials so that the blend exhibits uniform physical properties throughout the blended volume.

The term “contour” refers to an outline, particularly of a curving or irregular figure (e.g., residual limb), in three-dimensional space.

The term “custom-fit” refers to a personalized or individualized device (e.g., liner) concerning shape and size. A custom-fit liner implies modification of characteristics according to the unique dimensions of a residual limb.

The term “residual limb” refers to the portion of an arm or leg that remains attached to a person's body after accidental or intentional amputation or genetic deformity.

Unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value, or condition that deviates by or less than 10% of the stated amount, value, or condition.

It will be understood that unless a term is defined to possess a described meaning, there is no intent to limit the meaning of such term, either expressly or indirectly, beyond its plain or ordinary meaning.

### DETAILED DESCRIPTION OF VARIOUS EMBODIMENTS

A better understanding of different embodiments of the disclosure may be had from the following description and accompanying drawings in which reference characters refer to like elements.

While the disclosure is susceptible to various modifications and alternative constructions, certain illustrative embodiments are described in the drawings below. It should be understood, however, that there is no intention to limit the disclosure to the embodiments disclosed, but on the contrary, the invention covers all modifications, alternative constructions, combinations, and equivalents falling with the spirit and scope of the disclosure.

Embodiments described herein relate to custom suspension liners. These embodiments can be fabricated directly on a residual limb or on a mold by a prosthetist to fit each amputee's unique limb shape, prominent bony limbs, limbs with sharp or irregular contours, a high level of sensitivity, and/or significant scarring such as skin grafts. In embodiments where the suspension liner is fabricated directly on a mold, the liner may be fabricated in a central fabrication center.

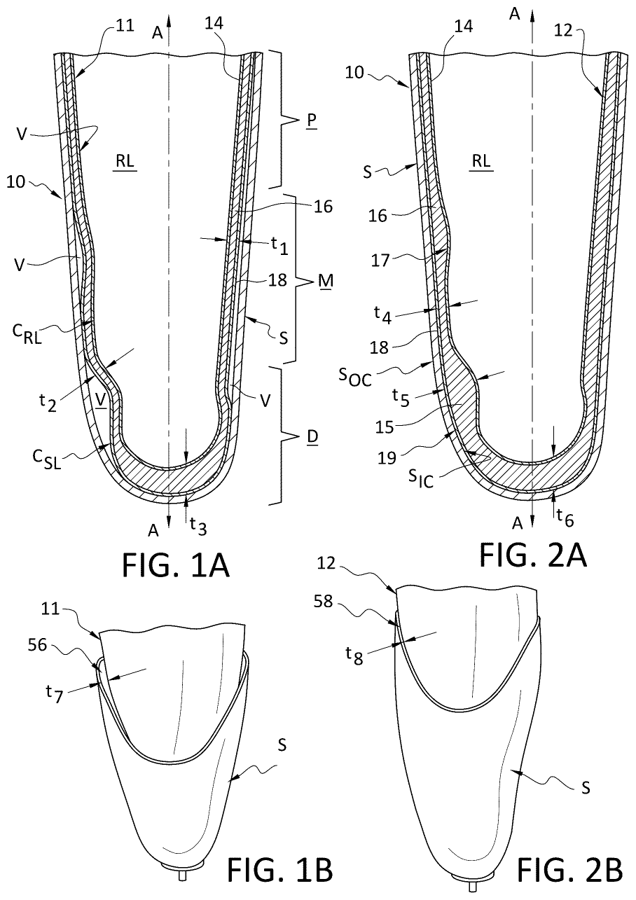

According to FIG. 1A, a suspension liner for a prosthetic system 10 may be made with a socket S. An open-ended, tubular-shaped liner blank 11 is provided, which forms the basis with a moldable material inserted therein to define the suspension liner, as depicted in FIG. 6. The liner blank 11, and hence the fabricated suspension liner 12, has a longitudinal axis A-A extending between distal and proximal regions D, P, with a middle region M located between the distal and proximal regions D, P. The distal and proximal regions D, P include corresponding distal and proximal ends.

FIG. 1A illustrates a prosthetic system 10 including a generally conical socket S and a liner blank 11 arranged to become the suspension liner 12. The liner blank 11 tightly fits about the residual limb, leaving no gap between an inner layer 14 and the residual limb RL. Likewise, as part of the tight fit, the elasticity of the liner blank 11 compresses about the residual limb RL due to the liner blank 11 elastically distending over the residual limb RL so that the liner blank 11 does not shift or migrate while donned on the residual limb RL.

FIG. 1A illustrates the tightly fitted liner blank 11 as corresponding to a contour or shape CRL of a residual limb RL, irrespective of the socket S, which surrounds the liner blank 11 and residual limb RL. Before fabricating the suspension liner 12 with the liner blank 11 (i.e., adding and curing a moldable material into the liner blank), FIG. 1A illustrates how the liner blank 11 contours closely to the residual limb RL in contrast to a generally conical shape of a socket S. The residual limb RL and liner blank 11 form a plurality of voids V that will be filled as the suspension liner 12 is formed by the injection of the moldable material, which causes the liner blank 11 to distend radially outwardly relative to the axis A-A.

As shown in FIG. 1A, the liner blank 11 includes an inner layer 14 arranged in shape to follow the contour CRL of the residual limb RL in such aforementioned tight fit, whereby the inner layer 14 forms an inner periphery 17 of the eventually fabricated suspension liner 12 to interface with the skin of the residual limb RL directly. In addition, the liner blank 11 includes an outer layer 18, forming an outer periphery 19 of the suspension liner 12. The outer periphery 19 defines a shape different from the shape of the inner periphery 17 once the suspension liner 12 is fabricated however, before fabricating the suspension liner 12, the outer periphery 19 generally or substantially follows the same contour as the inner layer 14, which no more than a 10% deviation therefrom, and particularly no more a +/−5% deviation from the contour of the inner layer 14 at least in the middle region M of the liner blank, and hence residual limb RL.

A middle layer 16 extends between the inner and outer layers 14, 18, and has a generally uniform thickness t1, t2 before fabrication of the suspension liner, with the thickness having no more than a +/−10% deviation between distal and proximal ends D, P of the liner blank 11, and particularly no more a +/−5% deviation. However, the distal end itself may be 1-4 times thicker than the middle layer, and more preferably 2-3 times thicker to offer sufficient cushioning for the distal end of a residual limb. Contrariwise to the liner blank 11, the definitive suspension liner 12 in FIG. 2A exemplifies the middle layer 16 as defining at least one variable thickness t4, t5 to accommodate the different shapes of the inner and outer peripheries 17, 19 of the suspension liner 12. The distal region D of the liner blank 11 may have a distal thickness t3 greater than a thickness t1, t2 in the middle region M, such that the distal thickness t3 is preformed of such certain thickness to assure sufficient cushioning of the fabricated suspension liner 12.

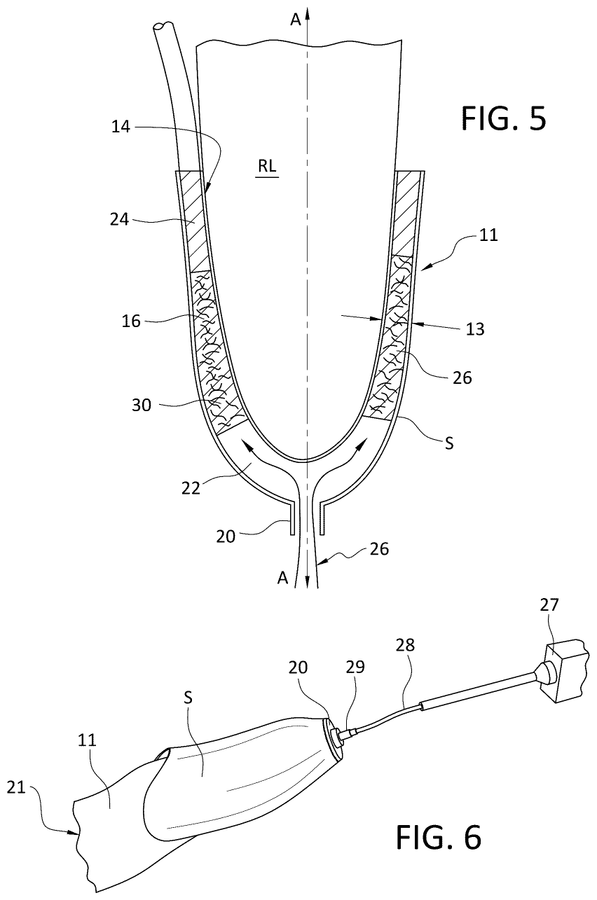

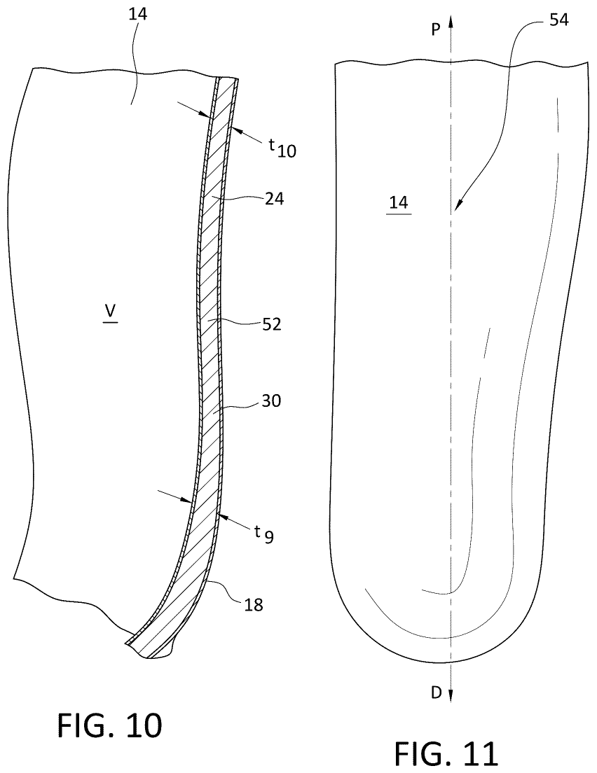

The liner blank 11 includes a casting core 30 (see FIGS. 5 and 10) disposed of in the middle layer 16. The casting core 30 is adapted to elastically and radially distend relative to the axis A-A. It provides a reinforcement means for the moldable material to be eventually injected into the liner blank 11. The casting core 30 is a fiber reinforcement constituent to the middle layer 16. In contrast, the moldable material 26 serves as a matrix material constituent to the casting core 30, thereby the casting core 30 and the moldable material 26 forming a composite of the middle layer 16. The casting core 30 and the moldable material 26 are chemically and structurally compatible materials integrated with the inner layer 14 and the outer layer 18.

In a variation, the liner blank 11 is devoid of the casting core 30, and the middle of the liner blank 11 is defined as a void between the inner and outer layers 14, 18, providing a space to inject the moldable material 26 therein for forming a standard solid thickness. Likewise, the middle layer 16 with the casting core 30 may include at least one section having a solid structure 24 (FIG. 5) devoid of the casting core 30 and comprising the moldable material 26.

The middle layer 16 may define at least one compensating area 15. The compensating area 15 may have at least one thickness t5 tapering in thickness and located in a middle portion M between the proximal and distal portions P, D of the liner relative to the axis A-A of the liner 12. The compensating area 15 may generally correspond in shape to a periphery of the void V before fabrication of the suspension liner to thereby fill the void V and offer a tight fit of the suspension liner 12 when donned on the residual limb RL and against the socket S, as illustrated in FIG. 2A. Further, as shown in FIG. 2A, the outer periphery 19 of the suspension liner corresponds in shape to a socket inner contour SIC of a socket S.

The inner layer 14 and the outer layer 18 may each have a substantially uniform thickness at or less than +/−10% deviation in the middle section M. Alternatively, at least when provided in the liner blank, the inner layer 14 and the outer layer 18 may have a uniform thickness of at most +/−2% deviation within the middle section M. The inner and outer layers 14, 18 may have non-uniform thicknesses in the distal or proximal D, P regions amounting to more than +/−10% deviation.

The liner blank 11 may be provided with a conventional prosthesis connecting element or may be provided with a continuous distal end, as in U.S. Pat. No. 8,597,368, granted on Dec. 3, 2013, and incorporated herein by reference.

FIGS. 1B and 2B illustrate the difference in the fit of the liner blank 11 with the socket S and the definitive suspension liner 12 with the socket S (once formed to custom-fit the residual limb and correspond to the socket). A thickness t7 illustrates a noticeable gap 56 between the socket S and the liner blank 11 in FIG. 1B. A thickness t8 illustrates a non-discernable gap 58 between the suspension liner 12 and the socket S. The thickness t7 has a preferable magnitude of being greater than 1 mm. In contrast, the thickness t8 has a magnitude of or less than 1 mm.

Generally, the fabricated suspension liner may have a structure resembling any embodiments discussed or incorporated into U.S. Pat. No. 9,788,977, granted Oct. 17, 2017, and U.S. Pat. No. 11,369,496, granted Jun. 28, 2022, incorporated herein by reference.

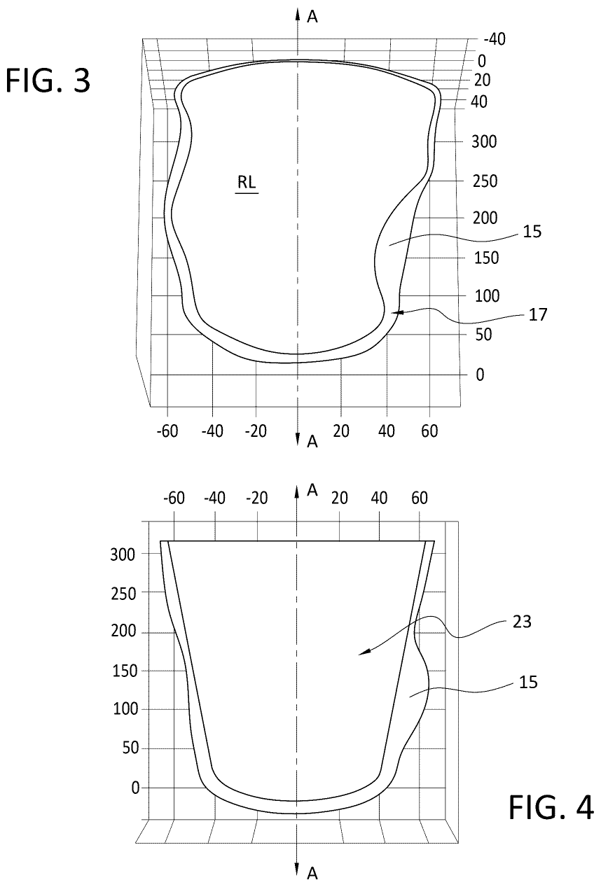

FIG. 3 illustrates a cross-sectional view of a graphically designed suspension liner 12 for stocked or “off-the-shelf” molds. The residual limb RL can be scanned using a 3D scanner to obtain a graphical representation or 3D model of the residual limb RL. The graphical representation includes the desired shape of the liner 12 to fill voids V, including undercuts or invaginations, and introduce at least one compensating area 15. Forming a custom liner 12 with an off-the-shelf mold avoids costs and saves time for manufacturing a customized mold.

FIG. 4 illustrates a cross-sectional view of a graphically designed suspension liner 12 formed on a standard-shaped mandrel 23. The liner 12 is inverted such that the compensating area 15 radially protrudes from the longitudinal axis A-A. This method avoids the costs of custom-made mandrel manufacturing and eliminates the need to store the mold.

FIGS. 5, 7, and 8 provide exemplary views of the casting core 30, which includes a network of filaments 32 with interstices 34 defined between the filaments 32. The filaments 32 may be formed by an elastic polymeric material compatible with the moldable material 26. The casting core 30 may be formed and possess structure according to the filaments and components formed in U.S. Pat. No. 10,271,968, granted Apr. 30, 2019, U.S. Pat. No. 11,312,071, granted Apr. 26, 2022, U.S. Pat. App. Publ. No. 2022/0314540, published on Oct. 6, 2022, and U.S. Pat. App. Publ. No. 2021/0137708, published on May 13, 2021, each of which is incorporated herein by reference. Other suitable filament arrangement may be envisioned that enable other techniques, kits and structure, and methods of the disclosure to be performed. Thus, the depicted filament arrangement is merely exemplary of one of many configurations of filaments.

The casting core 30 preferably forms a circumferential sleeve extending at least within the middle region M of the suspension liner 12 and may have a closed distal end 33. Due to the network of filaments 32, the casting core 30 is arranged to expand radially relative to the axis A-A. More specifically, the casting core 30 is arranged to radially expand locally depending on a quantity of moldable material 26 located thereat, such that the casting core 30 expands a first thickness t4 within the middle region M and a second thickness t5 adjacent to the first thickness t5 within the middle portion and radially relative to the axis A-A.

The casting core 30 and the moldable material 26 include at least one compatible polymeric material such that the moldable material 26 bonds to the casting core 30. The casting core 30 and moldable materials may be those discussed in U.S. Pat. No. 6,485,776, U.S. Pat. App. Publ. No. 2022/0314540, and U.S. Pat. App. Publ. No. 2021/0137708. The casting core 30 consists of staggered lines, loops, or other shapes to enable radial expansion from the longitudinal axis A-A. In addition to shape, the expandability of the casting core 30 is defined by the density of the casting core 30, the viscosity of the moldable material 26, and the pressure and speed of the injection of the moldable material 26 (e.g., the greater pressure present during injection of the moldable material 26, the greater the casting core 30 expands in the radial direction from the longitudinal axis A-A).

FIG. 9 shows an embodiment of a distal end 33 for a casting core 30, such that the distal end 33 forms a distal end cap 36 having at least one side relative to the axis A-A, either being an inner side or an outer side. The distal end cap 36 forms at least two ribs 40 and a channel 38 defined between the at least two ribs 40 for facilitating transmission of moldable material therethrough. The distal end cap 36 preferably forms a closed distal end 42.

The at least two ribs 40 include a first rib 44 extending distally toward the distal end 42 than a second rib (46). At least one of the first and second ribs 44, 46 has a proximal limit 48 located more proximal than a proximal limit 50 of another one of the first and second ribs 44, 46. The distal end of the first and second ribs 44, 46 tapers in width to facilitate the transit of moldable material in a proximal direction, and the proximal limits 48, 50 of the first and second ribs 44, 46 widens to impede at least some of the moldable material to urge retention and collection of moldable material 26 at the distal region D of the suspension liner 12. Thus, while the embodiment of the casting core 30 in FIG. 8 enables greater radial expansion of the casting core 30 from the longitudinal axis A-A, the embodiment in FIG. 9 enables the faster material flow of the moldable material 26 through the casting core 30 in a direction parallel to the longitudinal axis A-A.

FIG. 10 illustrates exemplary cross-sections of the suspension liner 12, particularly corresponding to FIG. 5. The silicone material 52 impregnates the casting core 30 and continuously extends into the solid section 24 without interruption. By “continuously extending,” the same silicone material 52 used as the moldable material 26 extends without interruption as a solid mass from the casting core into the solid section 24, albeit with the solid section 24 omitting the casting core 30. While non-limiting, the silicone material 52 impregnating the casting core 30 has a thickness t9 that may be greater than a thickness t10 of the solid section, or at least different therefrom. The impregnation comprises filling the interstices of the casting core 30, and the silicone material 52 bonding to the filaments 32, such that the filaments 32 and the silicone material 52 are inseparable from one another and define a permanent bond with the silicone material 52 and the filaments 32 (which may likewise be formed from a silicone material) interlocking chemically and mechanically with one another.

FIG. 11 depicts a suspension liner 12, or a liner blank 11 that may include an orientation line 54 extending between proximal and distal regions P, D. The orientation line 54 may be located on the inner layer 14, or may preferably be on the outer layer 18, such as a textile covering, to enable the user guidance which donning the liner, such as rolling it on the residual limb. The orientation line 54 may or may not correspond exactly to the axis A-A as the orientation line 54 is arranged relative to a shape of an inner periphery of the suspension liner and the residual limb RL.

According to the embodiments of the casting liner and hence fabricated suspension liner, FIGS. 2A and 5 depict a prosthetic system 10 including a fabricated suspension liner 12 from the liner blank 11 and a moldable material 26, and a prosthetic socket S. The suspension liner 12 is arranged to custom-fit to a contour or shape CRL of a residual limb RL and may possess any of the characteristics of the embodiments above herein. The socket S may be of any prosthetic socket, including an adjustable socket system, as in U.S. Pat. No. 10,806,607, granted on Oct. 20, 2020, or a conventional rigid socket as in U.S. Pat. No. 6,923,834, granted Aug. 8, 2002, and U.S. Pat. No. 5,885,509, granted Mar. 23, 1999, each of which is incorporated herein by reference.

A method and variations thereof are provided for making a custom-fitted suspension liner 12 for a residual limb RL, according to any of the embodiments described herein. Referring to FIGS. 1A, 5, and 6, the method generally involves placing a liner blank 11 on a residual limb RL. The liner blank 11 includes an inner layer 14 and an outer layer 18 separated by a gap 13. A moldable material 26 is injected into the liner blank 11 to fill the gap 13. The moldable material 26 bonds to both the inner periphery 17 of the inner layer 14 and the outer layer 18. Thus, the moldable material 26 is compatible with the materials of the inner layer 14 and the outer layer 18 to ensure bonding between layers 14, 18.

The injected moldable material 26 could be thermoformable, so the user will benefit from silicone against the skin of the residual limb RL while also obtaining the benefits of thermoformable material. A thickness of the middle layer 16 or gap 13 may be substantially uniform along the void V before injecting the moldable material 26. The moldable material 26 is molded to contours CRL of the residual limb RL by the pressure of the socket S and the residual limb RL, essentially sandwiched therebetween, to form a definitive suspension liner 12 into a shape of a residual limb RL, inclusive of filling any voids initially present between the socket S and the residual limb RL and liner blank 11. The moldable material 26 of the middle layer 16 chemically bonds to the inner layer 14 and the outer layer 18 to form the suspension liner 12.

The method may initially include donning a socket S over the liner blank 11 on the residual limb RL before injecting the moldable material 26. To simplify the socket S, it may be constructed of a generally conical shape, such that any voids formed between the outer layer 18 and the socket S before injecting the moldable material 26 are filled. By taking up variances between the residual limb and socket in the suspension liner, the process for forming the socket may be reduced, permitting less customization of the socket.

As noted, a thickness of the middle layer 16 or gap 13 may be substantially uniform along the void or plurality of voids V before injecting the moldable material 26. Upon injecting the moldable material 26, the middle layer 16 defines a thickness t5 at the compensating area 15 greater than a thickness adjacent t4 to the compensating area either in a proximal or distal direction relative to the axis A-A. Upon injecting the moldable material 26 under pressure into the gap 13, the moldable material 26 increases the gap 13 to distend toward the socket S and fills the at least one void V.

The moldable material 26 forms a solid mass of material to define a middle layer 16 of the definitive liner 12 and fill the gap 13. The definitive suspension liner 12 may have a variable thickness t4, 5, t6 extending between distal and proximal regions D, P of the definitive liner 12, including at least one compensating area 15 defining a non-uniform thickness and shape corresponding to the at least one void V. The definitive suspension liner 12 has an outer periphery 19 forming a generally conical shape about a central axis A-A and an inner periphery 17 having a generally a non-uniform shape relative to the generally conical shape of the outer periphery 19.

The outer periphery 19 is formed to exactly or near correspond in shape to a shape of an inner volume of a prosthetic socket S by limiting any gaps or voids. The inner periphery 17 is formed to correspond to the shape of the residual limb RL.

FIG. 6 illustrates the moldable material 26 as being injected into a distal end 22 of the liner blank 11 between the inner and outer layers 14, 18. The liner blank 11 may include a distal reinforcement 20 extending into the distal end 22 into which the moldable material 26 is inserted. The distal reinforcement 20 may be configured and dimensioned as an umbrella (e.g., distal reinforcement 120) or as a socket adapter and may also comprise an inlet connector 29. The moldable material 26 may terminate, extending through and filling the gap 13 at a proximal section between the inner and outer layers 14, 18 as it cures or whereat pressure from injecting the moldable material 26 wanes due to being further away from the inlet. In an embodiment, the liner blank 11 is printed with a lattice mesh support structure having a thin inner layer 14, outer layer 18, and inlet connector 29 at the distal end of the liner blank 11 for injection of a moldable material 26, the moldable material 26 being silicone. In an embodiment, the inlet connector 29 may be threaded or embedded with the distal reinforcement 20. The inlet connector 29 may comprise a locking pin to engage with the socket S.

As discussed, a casting core 30 may be located within at least a portion of the gap 13 at least between distal and proximal regions D, P of the liner blank 11. The casting core 30 is radially and axially elastic, with the inner and outer layers 14, 18 having surfaces secured to the casting core 30. The moldable material 26 impregnates the casting core 30 such that the casting core 30 distends at least radially outwardly relative to the axis A-A as the moldable material 26 is injected into the gap 13.

The liner blank 11 may define a substantially uniform thickness t1, t2 between proximal and distal portions thereof either due to the casting core thickness or positioning of the inner and outer layers 14, 18 relative to one another. The distal end 22 of the liner blank 11 may have a greater thickness t3 than the thickness t1, t2 between the proximal and distal portions of the liner blank 11. Such greater thickness t3 may be preformed by the casting core or a more generous gap between the inner and outer layers 14, 18.

FIG. 6 illustrates the method wherein the socket S includes a distal reinforcement 20. An injection tube 28 connects a pump 27, including uncured moldable material and is arranged to inject uncured moldable material into the liner blank 11 through the inlet connector 29. The inlet connector 29 may couple the distal reinforcement 20 to the injection tube 28. In an embodiment, the inlet connector 29 may comprise a locking element (e.g., threads or locking pin). The inlet connector 29 may be configured as a base component to engage with the prosthetic socket S. The liner blank 11 may be worn on a residual limb, and a socket S is donned over the liner blank 11 when the moldable material 26 is injected therein, or a positive mold 21, as in FIG. 6, may have a shape of the residual limb dons the liner blank 11 when the moldable material is injected into the liner blank.

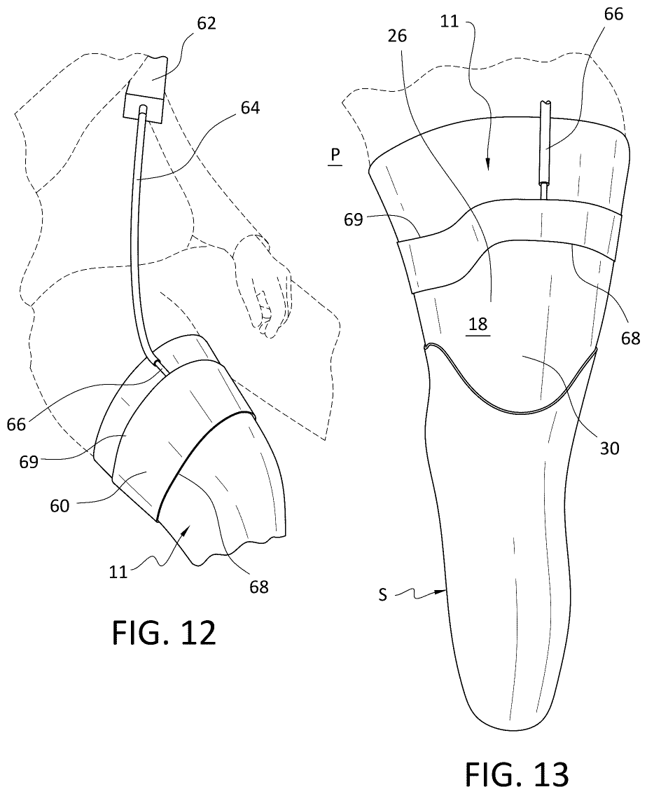

The injection point (e.g., inlet 20) seals itself when the moldable material cures. A plug or a valve may be used to disconnect the pump. During manufacturing, the most proximal part of the liner is sealed off by a blocking band, as discussed in connection with FIGS. 12 and 13, such as a thick silicone line connecting the inner skin layer and the outer textile layer by flooding part of the liner blank with silicone.

The inner layer 14 of the liner blank 11 may be formed in part of a composite elastic material. The outer layer 18 may comprise an elasticized fabric layer having an inner surface coated with a material, such as a thin continuous coating of silicone elastomer partially embedded in the fabric layer, compatible with the moldable material at least up to its distal end area, where a distal end cap is embedded therein a rigid prosthetic connector formed, for example, of aluminum or other metal, or rigid plastic such as Nylon, is provided.

Therefore, the fabricated suspension liner includes the inner, middle, and outer layers, such that the entire assembly of the inner, middle, and outer layers are at least freely, radially, and elastically distensible. Therefore, the liner blank and suspension liner may be formed as a closed-ended tapered tubular element, as is conventional for such suspension liners. The distal end cap joins the prosthetic connector to the suspension liner while providing a cushioning and stabilizing surface at the distal end of the liner. The prosthetic connector preferably includes a threaded aperture for providing access to a threaded prosthetic pin connector in a manner well-known in the art.

In a preferred example, the moldable material may be formed principally of a silicone elastomer obtainable from NuSil Technology of Carpinteria, Calif. under Product Designation CF13-2188. For a fuller description of the silicone material, reference may be made to U.S. Pat. Nos. 6,136,039 and 6,485,776. Further, the casting core may be removed, and other reinforcing materials may be used, such as fabric or other impregnable substrates, or yet further, a composition of elastomeric material may be selected that can be injected, molded, and cured without a reinforcement.

FIGS. 12 and 13 illustrate the step of placing a blocking band 60 about at least a portion of the circumference of the liner blank 11 at either the middle or distal regions M, D of the liner blank 11 to limit transit of the moldable material in a proximal direction. The blocking band 60 may extend circumferentially about the outer layer 18 of the liner blank 11. Alternatively, the band may not block per se, but be placed on the liner to absorb injected moldable material in the event there is seepage from the liner.

For example, a tube 64 may extend to an injection device 62, such as a siphon pump or syringe, from the proximal region P of the liner blank 11 to siphon air from the middle layer 16 of the liner blank 11 as the moldable material is injected from the distal region D into the middle layer 16, to facilitate impregnation of the moldable material into the casting core. The band may be absorbent to absorb the moldable material in case the outlet at the tube is not properly sealed. The syringe may apply a vacuum by sucking trapped air out of the liner blank. The liner blank 11 may include a duct 66 from the proximal region P and extend to the middle region M to communicate therewith. The duct 66 may be arranged to couple to the tube 64.

As opposed to the absorbing band, the blocking band 60 may have distal and proximal boundaries 68, 69, and correspond to the solid section, such that placement of the blocking band includes adding the blocking band 60 proximally from the casting core 30. The blocking band may halt the flow of the moldable material and let trapped air escape, such as by the syringe.

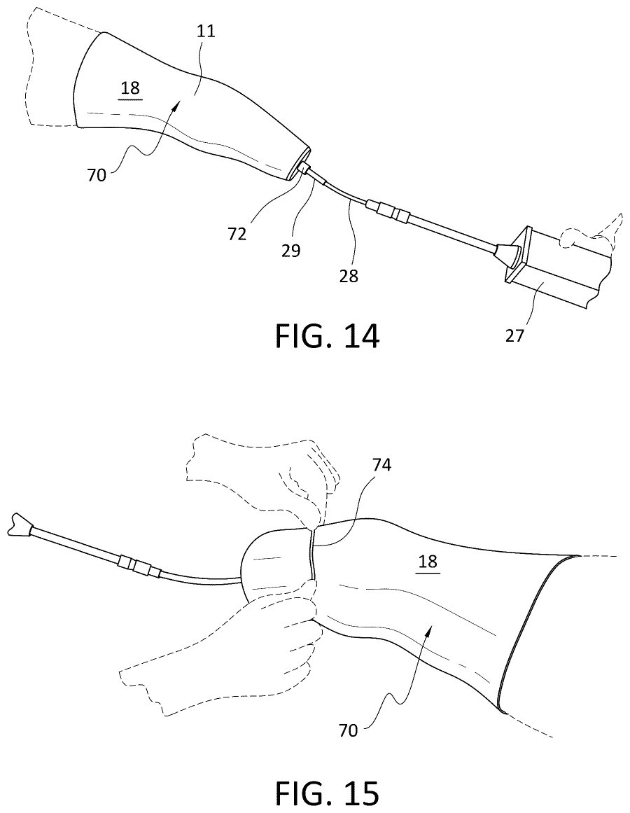

FIGS. 14 and 15 show that the suspension liner 70 may be directly cast onto a residual limb without a socket surrounding at least a portion of the liner blank 11. The method may include injecting the moldable material by a pump 27 through a tube 28 via the distal end of the liner blank 11 at an inlet connector 72. The inlet connector 72 may be configured as a base component and comprise a locking element (e.g., threads or locking pin) to engage with the distal reinforcement 20. A scraper 74 may be applied to the liner blank 11 once the moldable material is injected. The scraper 74 may be applied to the outer layer 18 to apply pressure onto the moldable material and the casting core.

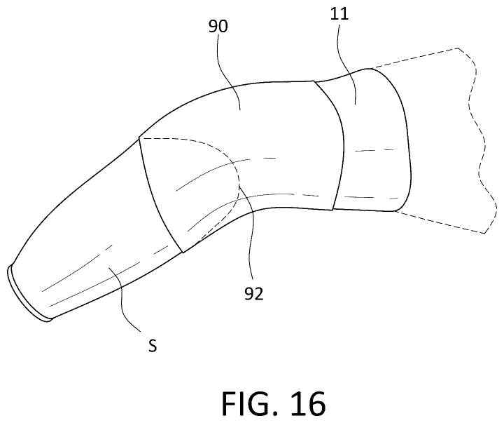

FIG. 16 illustrates the step of placing a pressure band 90 about at least a portion of the proximal region of the liner blank and the socket S to form a transition 92 of the middle layer between the interface of the socket and sleeve extending proximally therefrom.

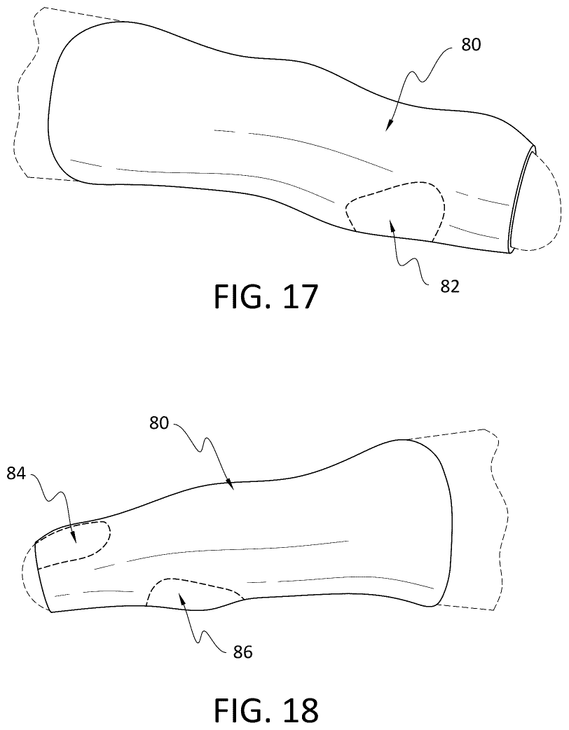

FIGS. 17 and 18 show the step of placing a casting sleeve 80 about a residual limb for forming a socket S. The method further includes placing volume control pads 82, 84, 86 between the casting sleeve 80 and the residual limb to form a more conically uniform socket S than a shape of the residual limb. The volume control pads 82, 84 may also be placed in areas of high pressure or sensitive parts of the residual limb where it is desirable to reduce pressure from the socket S by creating a void V which will later be filled with the moldable material while injecting the liner. The suspension sleeve may be formed according to any of the preceding steps to accommodate the voids between the socket S and the residual limb as a result of an omission of such volume control pads 82, 84, 86.

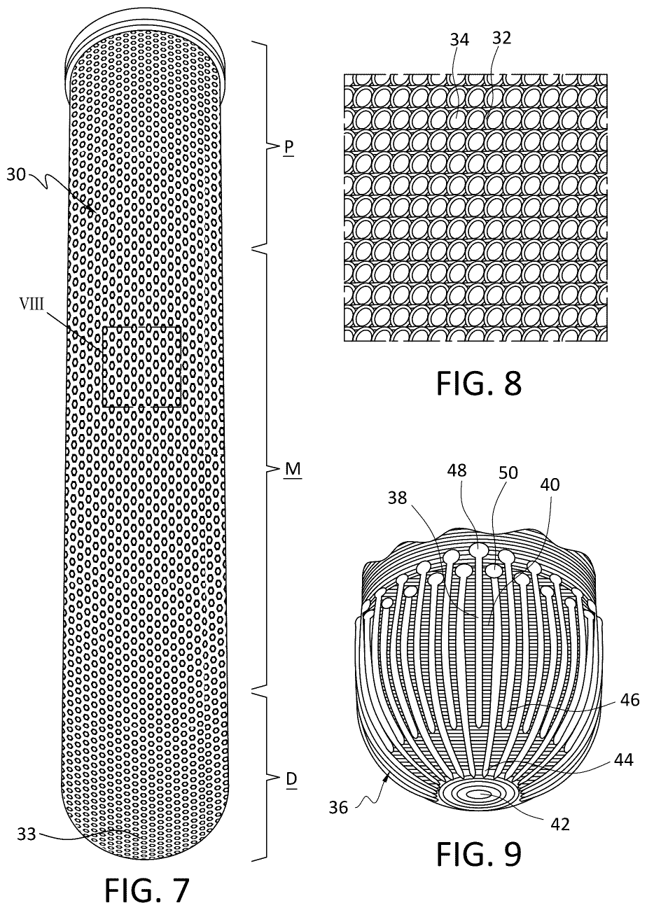

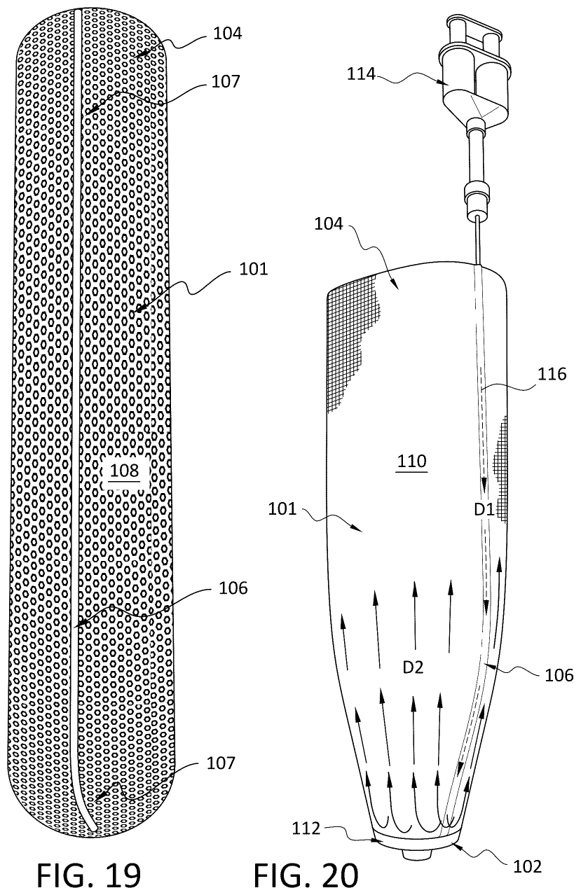

FIG. 19 illustrates a casting core 101, as in the embodiment of FIG. 7, having a closed distal end 102 and an open proximal end 104. A tube 106 is arranged along the surface 108. A tab 107 may be located proximate the open proximal end 104 and holds the tube 106 in place, whether permanently or temporarily. The tab 107 can enable the tube 106 to be removed once the moldable material has been injected into liner blank. Alternatively, the casting core 101 may be provided without the tab, and the tube 106 may be arranged to be pulled out of the liner blank.

The tube 106 is accessible for injecting moldable material into the tube and has an open end at the proximal end. The tube 106 extends to the distal end 102 and opens at the distal end 102 so that the moldable material dispenses at the distal end 102 of the casting core 101. Embodiments with the tube are preferably provided for prosthetic liners that are devoid of a liner with no distal holes or in cushion liners with no umbrella or other distal structure, which require a different injection point than the distal end.

FIG. 20 exemplifies a liner blank 100 having the casting core 101, or of any of the preceding embodiments, with a textile cover 110, as in preceding embodiments. The liner blank 100 has a tube 106 and FIG. 20 shows the injection device 114 attached at the proximal end 104 of the tube 106 with the moldable material 116 flowing in a first direction D1 toward the distal end 102. At the distal end 102, the moldable material 116 is redirected and flows in a second direction D2, opposite the first direction D1, toward the proximal end 104 (i.e., the silicone is urged upwards into the casting core 101). While there may be a greater aggregation of moldable material 116 at the distal end 102, the moldable material 116 reaches the proximal end 104. The liner blank 100 may also be provided with a distal reinforcement 112 arranged to reinforce a distal end 102 of the liner blank 100. In an embodiment, distal reinforcement 120 is an adapter configured and dimensioned to connect to a prosthetic knee and enable rotation of the flexed knee joint. The distal reinforcement 112 may comprise threads, pins, magnets, or other fastening means for connecting to a prosthetic knee.

At the end of injecting moldable material, the tube may be removed or remain in the blank or definitively formed prosthetic liner. The casting body may be formed with the tube being integrally formed with the casting body.

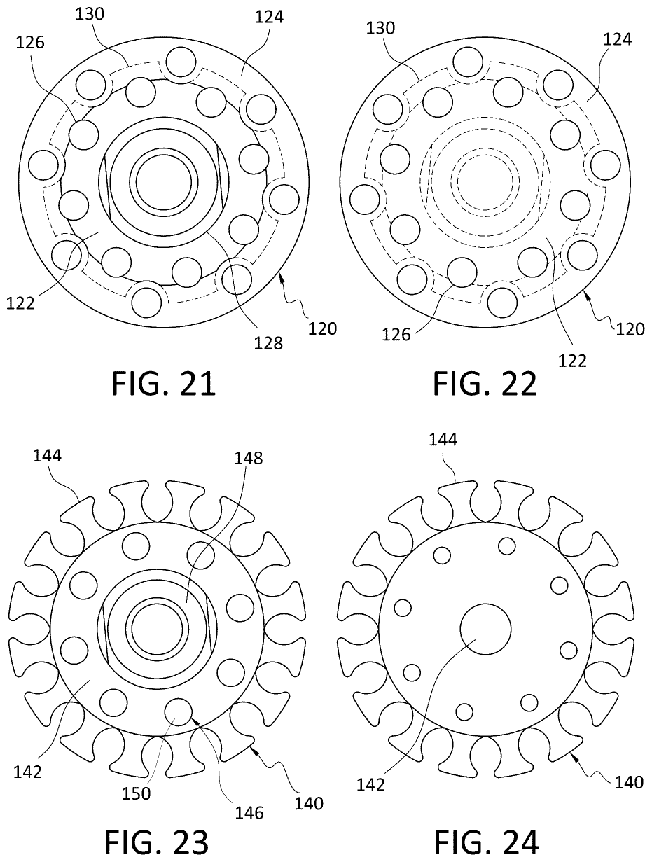

FIGS. 21-24 illustrate embodiments of a distal reinforcement or umbrella 120, 140 arranged to reinforce a distal end of a prosthetic liner. The reinforcement 120, 140 may include a locking element 128, 148 such as a threaded socket or other means, for coupling to prosthetic devices, such as a prosthetic socket. A custom liner conforms to the shape of the residual limb better than conventional off-the-shelf liners. A conventional umbrella is made of stiff materials (plastic and metal), which do not conform to the residual limb and can cause pressure peaks around the distal end. The distal end of the residual limb is often one of the most sensitive parts of the residual limb and having the umbrellas edges of softer material can be beneficial to the user, especially in the case of a custom liner.

According to the embodiment of FIGS. 21 and 22, the umbrella 120 includes a rigid base component 122 carrying the locking element 128, and a flexible edge component 124 interlocks or is molded over the periphery 130 of the base component 122. Material of the edge component 124 may interlock openings 126 formed by the base component 122, extending into and/or filling the interlock opening 126 to engage the base component 122 firmly. The edge component 124 may be molded about the base component 122 to create the depicted structure. The base component 122 may be formed from a metal or other structural material, and the edge component 124 may be formed from a polymeric material, such as an elastomer, which is more flexible than the base component 122.

FIGS. 23 and 24 depict a rigid base component 142 and a flexible edge component 144 that interlocks with openings 146 formed by the base component 142. The edge component 144 forms a plurality of tabs 150 configured and dimensioned to engage the openings 146. The base component 142 and edge component 144 are arranged similarly as in the embodiment of FIGS. 21 and 22, although the edge component 144 is formed definitively separate from the base component 142, and fits or snaps into the base component 142 via openings 146 and tabs 150.

In both of the aforementioned embodiments, the edge component 124, 144 is arranged to extend over the inner side of the reinforcement 120, 140 so as to effectively serve as a cup to the distal end of the liner. Indeed, the edge component 124, 144, therefore, extends over the inner side of the reinforcement 120, 140 and beyond a periphery (e.g., periphery 130) of the base component 122, 142 to provide for cushion and flexion, yet reinforcing the distal end of the liner.

Of course, it should be understood that not necessarily all objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

The skilled artisan will recognize the interchangeability of various disclosed features. In addition to the variations described herein, other known equivalents for each feature can be mixed and matched by one of ordinary skill in this art to construct a suspension liner in accordance with principles of the present invention.

Although this invention has been disclosed in the context of certain exemplary embodiments and variations thereof, it therefore will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above.

1.-20. (canceled)

21. A suspension liner for a prosthetic system including a socket, the suspension liner arranged to custom-fit to a contour or shape of a residual limb, the suspension liner comprising:
an inner layer arranged in shape to follow the contour of the residual limb, the inner layer forming an inner periphery of the suspension liner;
an outer layer forming an outer periphery of the suspension liner, the outer periphery defining a shape different from a shape of the inner periphery;
a middle layer extending between the inner and outer layers, the middle layer defining a variable thickness between proximal and distal end regions of the liner to accommodate the different shapes of the inner and outer layers of the suspension liner;
a casting core disposed in the middle layer, the middle layer including the casting core and a moldable material.

22. The suspension liner of claim 21, defines at least one section of the middle layer having a solid structure devoid of the casting core and comprising the moldable material.

23. The suspension liner of claim 21, wherein the middle layer is located proximal relative to a central axis from the casting core and comprising the moldable material.

24. The suspension liner of claim 21, wherein a distal end of the distal end region comprises a solid structure devoid of the casting core and comprising the moldable material.

25. The suspension liner of claim 21, wherein the middle layer defines at least one compensating area, the compensating area having at least one thickness tapering in thickness and located in a middle region between the proximal and distal regions of the liner relative to a central axis of the liner.

26. The suspension liner of claim 21, wherein the inner layer has a substantially or entirely uniform thickness.

27. The suspension liner of claim 21, wherein the outer layer has a substantially or entirely uniform thickness.

28. The suspension liner of claim 21, wherein the casting core includes a matrix formed by filaments with interstices defined between the filaments, the filaments being formed by an elastic polymeric material compatible with the moldable material.

29. The suspension liner of claim 21, wherein the casting core forms a circumferential sleeve extending at least within the middle region of the suspension liner.

30. The suspension liner of claim 21, wherein the casting core is arranged to radially expand relative to a central axis of the liner.

31. The suspension liner of claim 21, wherein the casting core is arranged to radially expand locally depending on a quantity of moldable material located thereat, such that the casting core expands a first thickness within the middle region and a second thickness adjacent the first thickness within the middle region and radially relative to a central axis.

32. The suspension liner of claim 31, wherein the casting core and the moldable material are selected from at least one compatible polymeric material such that the moldable material bonds to the casting core.

33. The suspension liner of claim 21, wherein the distal end forms a distal end cap having at least one side relative to a central axis, the distal end cap forming at least two ribs and a channel defined between the at least two ribs for facilitating transmission of moldable material therethrough.

34. The suspension liner of claim 33, wherein the at least two ribs include a first rib extending distally toward a distal end of the distal region than a second rib;
wherein one of the first and second ribs has a proximal limit located more proximal than a proximal limit of another one of the first and second ribs.

35. The suspension liner of claim 34, wherein the distal end of the first and second ribs tapers in width to facilitate transit of moldable material in a proximal direction, and proximal limits of the first and second ribs widens to impede at least some of the moldable material to urge retention and collection of moldable material at the distal region of the suspension liner.

36. A prosthetic system including a suspension liner and a socket, the suspension liner arranged to custom-fit to a contour or shape of a residual limb, the suspension liner comprising:
an inner layer arranged in shape to follow the contour of the residual limb, the inner layer forming an inner periphery of the suspension liner;
an outer layer forming an outer periphery of the suspension liner, the outer periphery defining a shape different from a shape of the inner periphery;
a middle layer extending between the inner and outer layers, the middle layer defining a variable thickness between proximal and distal regions of the liner to accommodate the different shapes of the inner and outer layers of the suspension liner;
wherein the outer periphery corresponds in shape to a socket inner contour of a socket, the socket defining a substantially uniform or entirety uniform thickness between the socket inner contour and a socket outer contour at least between proximal and distal regions of the socket.

37. A method for making a custom-fitted suspension liner for a residual limb, the method comprising the steps of:
placing a liner blank on a residual limb, the liner blank including an inner layer and an outer layer separated by a gap;
injecting a moldable material into the liner blank to fill the gap;
curing the moldable material to contours of the residual limb to form a definitive suspension liner into a shape of a residual limb.

38. The method of claim 37, further comprising the step of donning a socket over the liner blank on the residual limb before injecting the moldable material.

39. The method of claim 38, wherein at least one void is formed between the outer layer and the socket prior to injecting the moldable material.

40. The method of claim 39, wherein a thickness of a middle layer between the inner layer and the outer layer is substantially uniform along the void prior to injecting the moldable material;
wherein upon injecting the moldable material, the middle layer defines a thickness at a compensating area greater than a thickness adjacent the compensating area either in a proximal or distal direction relative to a central axis.
